(19) United States
(12) Patent Application Publication
(10) Pub. No.: US 2025/0375160 A1
(43) Pub. Date: Dec. 11, 2025

(54) CIRCUMAURAL HEADSET OR HEADPHONES WITH PHYSIOLOGIC SENSOR CUFF

(71) Applicant: LIGHTSPEED AVIATION, INC., Lake Oswego, OR (US)

(72) Inventors: Brian David FROST, Lake Oswego, OR (US); Allan SCHRADER, Lake Oswego, OR (US); Greg HARMS, Lake Oswego, OR (US); Trent AMPOMAH, Lake Oswego, OR (US)

(73) Assignee: LIGHTSPEED AVIATION, INC., Lake Oswego, OR (US)

(21) Appl. No.: 19/232,770

(22) Filed: Jun. 9, 2025

Related U.S. Application Data
(60) Provisional application No. 63/657,826, filed on Jun. 8, 2024.

(51) Int. Cl. A61B 5/00; A61B 5/1455; B64D 45/00; H04R 1/10

(52) U.S. Cl. CPC A61B 5/6803; A61B 5/14552; A61B 5/7282; A61B 5/741; A61B 5/746; B64D 45/00; H04R 1/1008; H04R 1/1041; H04R 1/1075; A61B 2503/22; A61B 2560/0266; A61B 2560/0443; A61B 2562/222; A61B 2562/227; H04R 2420/09

(57) ABSTRACT

A circumaural headset includes a circumaural assembly having a physiologic sensor integrated within a generally U-shaped cuff having a formable wire frame encapsulated in a molded material and secured by a connector to an earcup of the circumaural assembly. The U-shaped cuff includes formable side portions to grip corresponding sides of a circumaural ear seal with a top portion of the cuff connecting the formable side portions and extending across a face surface of the circumaural ear seal, which is secured directly or indirectly to the earcup. The physiologic sensor is removably physically connected by a strain-relief plug of the connector to a corresponding molded socket within a faceplate of the earcup and electrically connected to one or more processors programmed to process signals from the physiologic sensor and provide an audible alert via at least one of the circumaural assemblies in response to sensor data.

### CROSS-REFERENCE TO RELATED APPLICATIONS

This application claims the benefit under 35 U.S.C. § 119(e) of U.S. Application No. 63/657,826 filed Jun. 8, 2024, the disclosure of which is hereby incorporated by reference in its entirety.

### TECHNICAL FIELD

This disclosure relates to circumaural headsets, headphones, and earmuffs having a removable adjustable physiological sensor.

### BACKGROUND

Various types of sensors are being used to monitor personal physiological parameters related to health and/or performance during specified events or time periods, as well as during everyday activities. Monitoring of parameters such as heart rate, blood pressure, respiration rate, oxygen saturation, blood chemistry, blood flow, etc. under various environmental and use conditions presents numerous challenges in providing an acceptable sensor signal for processing. For example, motion artifacts generated by movement of the user and/or sensor during use may decrease accuracy of the resulting signal analysis results if not properly accommodated. Similarly, variation in positioning of the sensor relative to an expected placement, or movement during use may result in decreased accuracy. Changes in ambient conditions, such as variations in ambient light, sound, vibration, etc. may also contribute to noise in the sensor signal.

Physiologic sensors have been integrated with headsets, headphones, and earphones as the ear has been identified as being particularly amenable to photoplethysmography (PPG), or the optical volumetric measurement of blood flow, and similar optical measurements. Pulse oximetry sensors have been integrated into the cushion of circumaural headsets to measure blood oxygen saturation. Earphones, ear buds, headphones, and similar devices provide a convenient form factor that users are generally familiar with and comfortable with positioning of the devices.

### SUMMARY

In one embodiment, a circumaural headset includes a band connecting first and second circumaural assemblies. At least one circumaural assembly includes a physiologic sensor integrated within a generally U-shaped cuff having a formable wireframe encapsulated in a molded material and secured by a connector to an earcup of the circumaural assembly. The U-shaped cuff includes formable side portions to grip corresponding sides of a circumaural ear seal with a top portion of the cuff connecting the formable side portions and extending across a face surface of the circumaural ear seal, which is secured directly or indirectly to the earcup. The physiologic sensor is removably physically connected by a strain-relief plug of the connector to a corresponding molded socket within a faceplate of the earcup and electrically connected to one or more processors programmed to process signals from the physiologic sensor and provide an audible alert via a speaker of at least one of the circumaural assemblies in response to sensor data. In one embodiment, the cuff and associated earcup include registration or alignment indicators to provide a visual indication of the position of the cuff relative to the associated earcup. The cuff is configured to slide along the ear seal to position the physiologic sensor in contact with a user forward of a tragus of the user when the headset is worn. The ear seal may provide a resilient force to hold the sensor in contact with the user. In one embodiment, the physiologic sensor comprises a pulse oximeter that provides signals indicative of blood oxygen saturation and heart rate.

Various embodiments of a headset, headphones, or muff having an adjustable removable physiologic sensor may include earcups with additional components for active noise reduction (ANR), passive hearing protection, audio, and/or voice communications using wired or wireless technology. ANR applications may include at least one earcup having a driver, error (sense) microphone, an optional voice/speech microphone and/or an optional ambient noise microphone coupled to one or more controllers to provide ANR and voice/speech functions.

One or more embodiments of a headset may include an associated controller having a microprocessor in communication with a physiologic sensor mounted to at least one circumaural assembly. The sensor may be integrated within a removable formable cuff configured to slide along a face surface of an ear seal to move the sensor to a desired position and to be bent to grip sides of the ear seal to maintain a desired position on the ear seal. The ear seal provides a resilient force to maintain contact between the sensor and skin of the user while delivering a comfortable fit while wearing the headset. The controller may be programed to analyze signals from the sensor. In one embodiment, the controller is programmed to detect quality of signals provided by the sensor that may be affected by movement of the user or position of the sensor. The controller may also be programmed to provide feedback to the user during a positioning process to indicate relative strength or associated confidence level of signals provided by the sensor to facilitate best positioning of the sensor.

Embodiments according to the disclosure may include a headset comprising a circumaural earcup assembly having an earcup with a faceplate and an ear seal mounted to the faceplate, the earcup having a socket secured within an interior of the earcup and configured to receive a plug; and a cuff having a formable wireframe encapsulated in a molded material, the cuff including side portions formable to grip corresponding sides of the ear seal, the side portions connected by a center portion having an opening, the cuff including an integrated physiologic parameter sensor extending at least partially through the opening, the integrated physiologic parameter sensor configured for coupling by a connector to at least one processor programmed to process sensor data, the connector including a cable terminating in a plug configured to engage the socket in the earcup to removably secure the cuff to the earcup. The cuff may comprise an outer cuff integrally formed of unitary construction of molded silicone. The cuff may further comprise an inner cuff formed of molded silicone encapsulating the formable wireframe, the inner cuff may be fixedly secured to an underside of the outer cuff by an adhesive. The center portion of the outer cuff may taper from thicker to thinner toward upper and lower edges of the center portion. A center portion of the inner cuff may taper from thicker to thinner toward upper and lower edges of the center portion of the inner cuff. The physiologic parameter sensor may comprise a pulse oximetry sensor. The plug of the connector may comprise a retainer configured to cooperate with a complementary retainer of the socket in the earcup configured to provide mechanical coupling of the plug and the socket. The retainer of the socket may be integrally molded in the faceplate of the earcup. The physiologic sensor may comprise a circuit board. The inner cuff may include a molded inset configured to recess the circuit board such that the circuit board is sandwiched between the outer cuff and the inner cuff. The inner cuff may include a molded channel configured to recess a portion of the connector and sandwich the recessed portion of the connector between the inner cuff and the outer cuff. The headset may further include a controller and a speaker disposed within the circumaural earcup assembly and coupled to the controller. The controller may be programmed to generate voice alert signals for the speaker in response to signals from the integrated sensor indicating blood oxygen saturation of a user being below an associated critical threshold level for a programmable amount of time. The controller may be programmed to suppress alerts during takeoff. The controller may be programmed to suppress alerts until an altitude increase of a specified amount, such as 2500 feet is detected. The controller may be programmed to generate an average blood oxygen saturation value using only blood oxygen saturation measurements from the integrated sensor that are above a first fit threshold and below a second fit threshold. The controller may generate a voice alert to reposition the cuff if sensor measurements are not within the first and second fit thresholds for a programmable period of time or a programmable number of measurements. The faceplate of the earcup may include a plurality of sliding position reference marks and the side portion of the cuff may include a single alignment mark to facilitate positioning of the cuff relative to the faceplate. At least one controller may be programmed to provide feedback to a user in response to signal quality from the integrated sensor at different relative positions between the cuff and the faceplate to facilitate sliding positioning of the cuff along the ear seal to improve or maximize quality of sensor measurements.

Embodiments according to this disclosure may provide one or more advantages. For example, adjustable mounting of a physiologic sensor to a circumaural headset may allow the user to adjust the position of the sensor relative to the headset to improve signal to noise ratio and resulting accuracy and reliability of the sensor signal. The circumaural headset may provide isolation for the sensor to reduce the effect of environmental factors, such as ambient noise and light, on the sensor signals. Resilient mounting of a sensor may improve skin contact with the sensor during physical activity, while also improving comfort. Positioning of the sensor forward of the tragus within a designated target area using a circumaural headset/headphone provides limited location variability from person to person. An adjustable removable and formable cuff having an integrated physiologic sensor according to various embodiments facilitates user positioning of the sensor for proper placement using feedback from an associated embedded app or coupled mobile app that monitors sensor signal quality. Processing of sensor signals according to various embodiments provides validation of sensor measurements to monitor sensor data outputs and reduce or prevent excessive sensor placement warnings to the user or reporting false positives and negatives for the monitored parameter(s) associated with low quality measurements due to movement of the user.

The above advantages and other advantages and features will be readily apparent from the following detailed description of the preferred embodiments when taken in connection with the accompanying drawings.

### BRIEF DESCRIPTION OF THE DRAWINGS

FIG. 1 illustrates a representative circumaural headset having a physiologic sensor integrated into an adjustable formable cuff according to one or more embodiments.

FIG. 2 is a first view of a formable cuff sensor secured to an ear seal of a circumaural assembly according to a first embodiment.

FIG. 3 is a second view of the formable cuff sensor of the first embodiment.

FIG. 4 illustrates a faceplate of an earcup without an ear seal illustrating a formable cuff sensor of the first embodiment connected by a strain-relief plug to a corresponding socket of the faceplate.

FIG. 5 is a first view of a formable cuff sensor for a circumaural headset according to a second embodiment.

FIG. 6 is a second view of the formable cuff sensor of the second embodiment.

FIG. 7 is a third view of the formable cuff sensor of the second embodiment.

FIG. 8 is an exploded assembly view of components of a formable cuff sensor according to one or more embodiments.

FIG. 9 is a first view of a formable wireframe embedded within a formable cuff sensor according to one or more embodiments.

FIG. 10 is a second view of the wireframe of FIG. 9.

FIG. 11 is a simplified block diagram illustrating a control system for a circumaural headset having a formable cuff sensor with an integrated physiologic sensor according to one or more embodiments.

FIGS. 12A, 12B, 12C, 12D, and 12E illustrate a control process or algorithm for validating sensor data from a circumaural headset cuff sensor having an integrated pulse oximeter according to one or more embodiments.

FIGS. 13A and 13B illustrate a control process or algorithm for generating alerts using a circumaural headset based on data from a cuff sensor having one or more integrated physiologic sensors according to one or more embodiments.

### DETAILED DESCRIPTION

As required, detailed embodiments are disclosed herein; however, it is to be understood that the disclosed embodiments are merely representative, and the claimed subject matter may be embodied in various and alternative forms not explicitly illustrated or described. The figures are not necessarily to scale; some features may be exaggerated or minimized to show details of particular components. Therefore, specific structural and functional details disclosed are not to be interpreted as limiting, but merely as a representative basis for teaching one skilled in the art to variously employ the claimed subject matter.

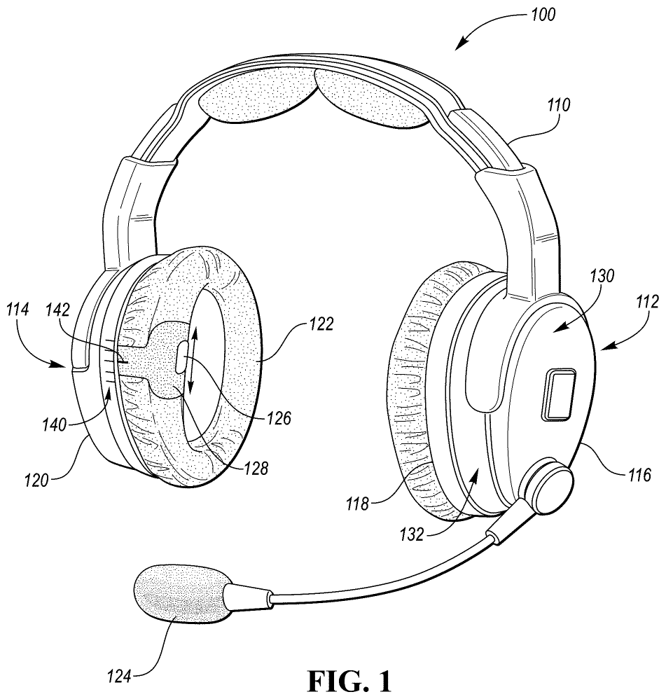

FIG. 1 illustrates a representative circumaural headset having an adjustable and removable physiologic sensor according to one or more embodiments. Headset 100 includes a band 110 connecting first circumaural earcup assembly 112 and second circumaural earcup assembly 114. Each earcup assembly 112, 114 includes an associated earcup 116, 120 and ear seal or cushion 118, 122. Each earcup 116, 120 includes a bottom portion 130 and a circumaural side portion 132. Headset 100 may include a microphone 124, which is implemented by a wired boom microphone in the representative embodiment illustrated. Headset 100 may include a wired connection to a control box (not shown) containing batteries, user controls, and one or more processors as described in greater detail herein. In other embodiments, headset 100 may communicate with an associated wireless microphone or with a wireless device having a microphone. When included, a microphone may be implemented with or without a boom, on a short boom, integrated into a wired connection, implemented by an optical comparator system, etc. Some embodiments do not include an associated microphone.

Headset 100 includes at least one biometric or physiologic sensor 126 integrated within a removable and slidably adjustable mount 128, which is formable to grip the sides of an associated ear seal or cushion 118, 122 and is removably secured to an associated one of the first 112 and second 114 circumaural earcup assemblies by a wired connector plug that engages a socket fixedly secured within an associated faceplate of the earcup. Various representative embodiments are described with reference to a biometric or physiologic sensor. However, those of ordinary skill in the art will recognize that sensor 126 may be implemented by various types of sensors that may employ chemical, electrical, and/or optical technology to detect various physiologic parameters in addition to providing detection or measurement of various environmental conditions as well as user characteristics and/or movements. In one embodiment sensor 126 is a multi-function sensor including signal processing electronics, memory, and a microprocessor/microcontroller to generate pulse oximetry data indicative of user blood oxygen level, heart rate, and user acceleration/motion. Multi-function sensor 126 may be implemented by the MAX32664 sensor hub and associated accelerometer and pulse oximeter available from Analog Devices, Inc. of Wilmington, MA, USA. As such, the representative embodiments described and illustrated are not limited to purely physiologic or biometric sensors, but may also include sensors such as acoustic sensors, accelerometers, and gyroscopes, for example.

As described in greater detail herein, the removable and slidably adjustable sensor mount 128 is configured to be movable along an associated ear seal or cushion to adjust a position of the sensor 126 relative to the earcup assembly 114 to position the sensor within a target region of the user in contact with the skin on the face of the user generally forward of a tragus of the user's ear. An embedded or linked app may be used to provide feedback to the user during a fit or positioning process to position the sensor 126 by sliding the mount 128 to different positions while monitoring signals provided by the sensor. Alignment marks 140, 142 on the earcup 120 and cuff mount 128 provide a visual indication for the user for reference during the positioning process and subsequent use after completing the positioning process. The adjustable cuff sensor includes an integrated wire frame so that after positioning the cuff sensor 128, the user may squeeze/bend the sides of the cuff to form to the sides of the ear seal and maintain the position of the cuff sensor during use. Any subsequent repositioning may be performed by unbending the sides of the cuff sensor and sliding along the associated ear seal during the positioning process.

While a single cuff sensor 128 is illustrated secured to the right ear seal in the representative embodiment of FIG. 1, other embodiments include a cuff sensor 128 positioned on the left ear seal and embodiments with cuff sensors on both left and right ear seals. Applications that use cuff sensors on both the left and right ear seals may use different sensors on each ear seal. For headsets having a connected wired control box, positioning of the cuff sensor 128 on the same earcup as the microphone 124 reduces the otherwise required wiring across the headband 110 between the earcups.

For embodiments employing a physiologic or multi-function sensor, sensor 126 may be implemented by any of a number of commercially available sensors that may be used to provide signals indicative of physiological parameters or characteristics of the user/wearer such as heart rate, blood pressure, respiration rate, oxygen saturation, blood chemistry, blood flow, etc. as previously described. Embodiments according to the disclosure may be used in aviation applications to provide alerts to pilots and passengers when blood oxygen saturation level (SpO₂) falls below designated thresholds.

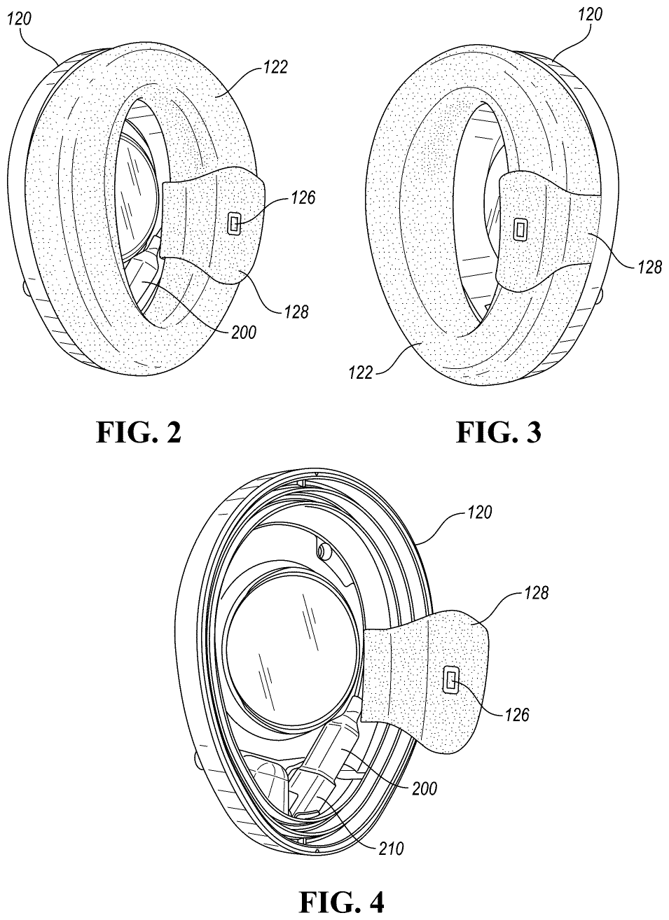

FIGS. 2-3 provide different views of a formable cuff sensor secured to an ear seal of a circumaural assembly of a headset or headphones according to a first embodiment. FIG. 4 illustrates a faceplate of an earcup without an ear seal illustrating a formable cuff sensor of the first embodiment connected by a strain-relief plug to a corresponding socket of the faceplate. With reference to FIGS. 2-4, formable cuff sensor 128 wraps around ear seal or cushion 122 and is slidingly positionable within a range limited by the length of the wired connector 200 that electrically connects a circuit board or sensor hub having sensor 126 to a socket contained within earcup faceplate 120. Connector 200 includes a multi-conductor flexible cable with a terminal plug configured to securely removably couple connector 200 to a corresponding socket 210 fixedly secured within faceplate 120. Socket 210 may be integrally molded within faceplate 210 or fixedly secured with an adhesive, by welding, or with one or more fasteners. As previously described, formable cuff sensor 128 contains an embedded wire frame so that the side portions of formable cuff sensor 128 may be squeezed or bent slightly inward to provide a frictional holding force between the cuff sensor 128 and the inside and outside perimeter of ear seal 122. The side portions of formable cuff sensor 128 may be bent slightly outward to reduce the frictional holding force and reposition or remove cuff sensor 128 as needed.

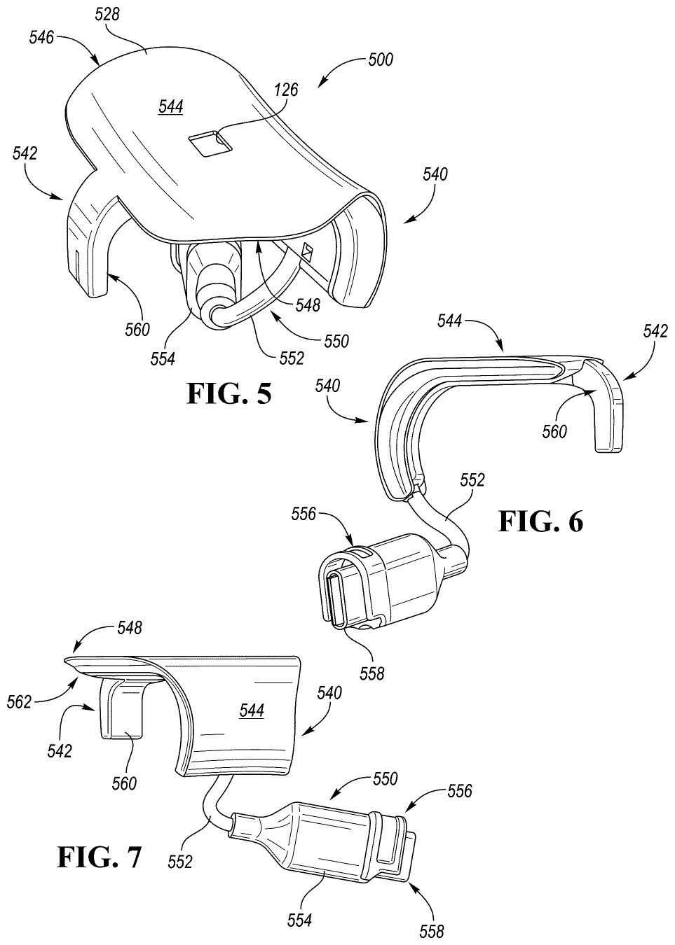

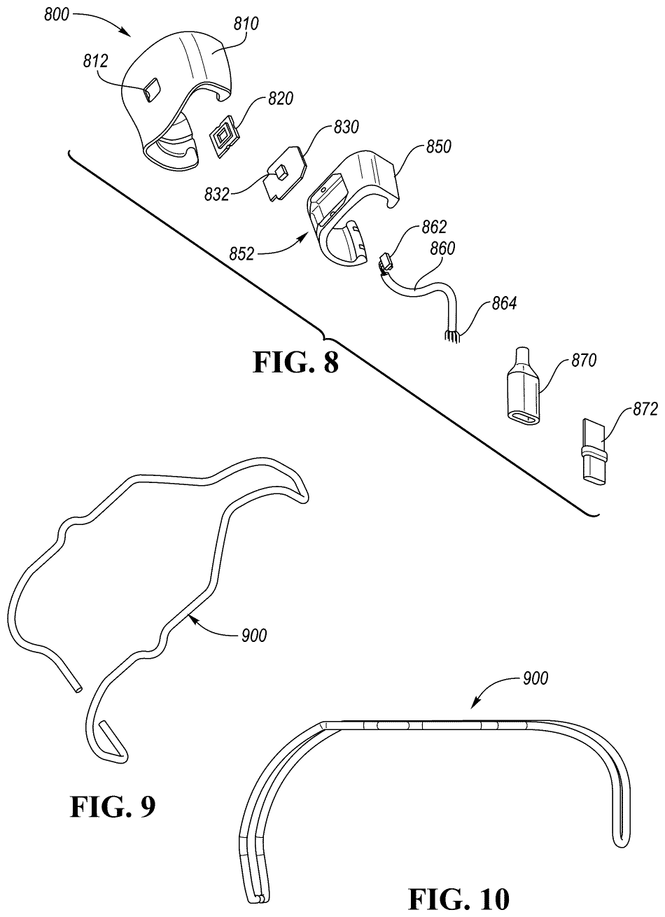

FIGS. 5-7 provide different views of a formable cuff sensor for a circumaural headset according to a second embodiment. Formable cuff sensor 500 includes an outer cuff 528 having a first leg or wing portion 540 connected to a second leg or wing portion 524 by a generally flat face surface 544. The bottom edge portion 546 and top edge portion 548 (as positioned on the ear seal) of surface 544 are tapered to reduce or eliminate any air gaps and associated sound leak paths between surface 544 and the user's head at the transitions from the associated ear seal and outer cuff 528. Outer cuff 528 is integrally formed of unitary construction. In one embodiment outer cuff 528 is molded silicone and includes a molded opening with a surrounding integrated gasket or seal for physiologic sensor 126. Formable cuff sensor 500 also includes an inner cuff 560 (best illustrated in FIG. 8) fixedly secured to outer cuff 528, such as by adhesive, for example. Inner cuff 560 encapsulates a formable or bendable wireframe 900 (see FIGS. 9-10) so that legs 540, 542 may be formed around an associated ear seal as previously described. In one embodiment, inner cuff 560 comprises silicone molded around wireframe 900 (FIGS. 9-10). Inner cuff 560 may also include a tapered top edge 562 and bottom edge (not specifically illustrated) to further reduce or eliminate any airgap at the transition between the cuff sensor/ear seal and the user's head.

Connector 550 includes a wire or cable 552 containing a plurality of conductors configured for electrically connecting a sensor hub or circuit board containing sensor 126 and related processing circuitry, memory, and microprocessor/microcontroller to one or more controllers of the circumaural headset. The circumaural headset may contain one or more controllers that may communicate with one another and be positioned in a single earcup, in both earcups, and/or in a control box connected to one or both earcups. One or more controllers in a mobile device may be wirelessly linked to one or more controllers of the headset and/or sensor hub.

As illustrated in FIGS. 5-7, a portion of wire or cable 552 is positioned and secured within a channel or slot formed between outer cuff 528 and inner cuff 560 with another portion extending out of the channel or slot and terminating in a strain-relief plug 554 that includes a retention feature or slot 556 at least partially surrounding electrical connector 558. Retention feature 556 may couple with a complementary retention feature formed on the corresponding socket fixedly secured or molded within the associated earcup of the headset to mechanically couple and removably secure the sensor cuff to the associated earcup.

FIG. 8 is an exploded assembly view of components of a formable cuff sensor according to one or more embodiments. Sensor 800 includes an outer cuff 810 formed of unitary construction from molded silicone. A silicone gasket or sensor seal 820 may be implemented as a separate component or may be integrated within the outer cuff molding around the sensor opening 812. A sensor hub or circuit board 830 includes at least one physiologic sensor 832 and associated hardware, firmware, software, memory, and electronic circuitry to generate physiologic data. Circuit board 830 may also include other sensors, such as accelerometers or gyroscopes, for example. In one embodiment, circuit board 830 is implemented by a MAX32664 sensor hub with a pulse oximeter and accelerometer commercially available from Analog Devices, Inc. of Wilmington, MA, USA.

Inner cuff 850 includes an embedded wireframe 900 (FIGS. 9-10) encapsulated within molded silicone. A molded inset and channel 852 is configured to accommodate sensor hub circuit board 830 and a portion of connector cable 860, which includes a plurality of conductors 864 extending between a first electrical connector 862 configured to electrically connect to circuit board 830 and integral sensor 832, and a second electrical connector 872 configured to electrically connect to a corresponding socket within the earcup of a circumaural headset. A strain-relief plug 870 may include a retention feature to removably secure the plug 870 to the corresponding earcup socket. The retention feature may include a twist lock, a click lock, locking tab, or similar feature to provide a mechanical connection in addition to the friction connection associated with the electrical connector. During assembly, connecter 862 is connected to circuit board 830, which is positioned within inset/channel 852 of inner cuff 850. A first portion of connector 860 is secured within the channel 852 with a second portion extending from inner cuff 850 to allow slidable positioning of the integrated cuff sensor along the ear seal as previously described. Inner cuff 850 is positioned within outer cuff 810 with sensor 832 extending within opening 812 to be flush or slightly above the surrounding face surface 844. Inner cuff 850 is fixedly secured to outer cuff 810 using adhesive or a similar method.

FIGS. 9 and 10 illustrate a formable or bendable wireframe for embedding within a formable cuff sensor according to one or more embodiments. Formable wireframe 900 facilitates bending of the legs/wings of the cuff sensor to grip the sides of an associated ear seal and maintain position of the cuff sensor as previously described. The wireframe 900 is formed of a ductile metal capable of repeated bending or forming while resisting breaking.

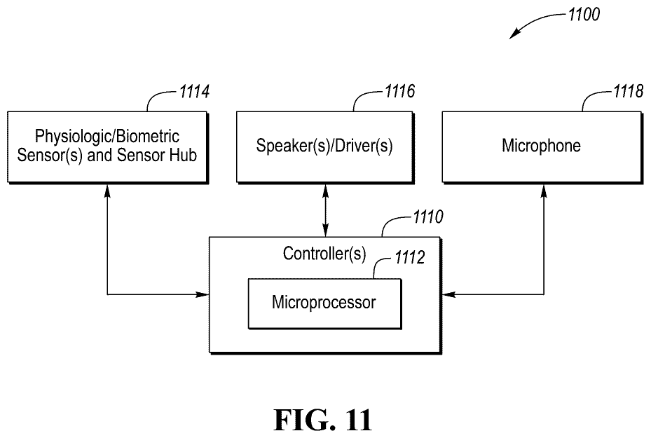

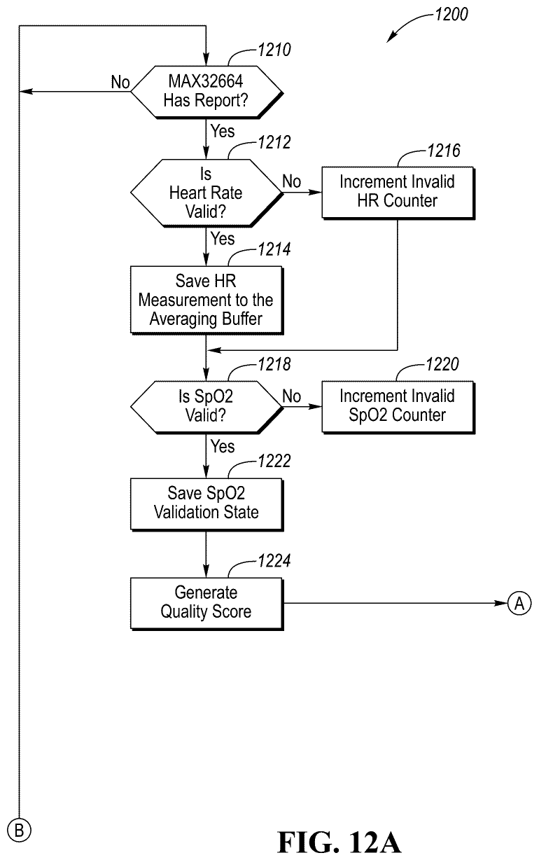

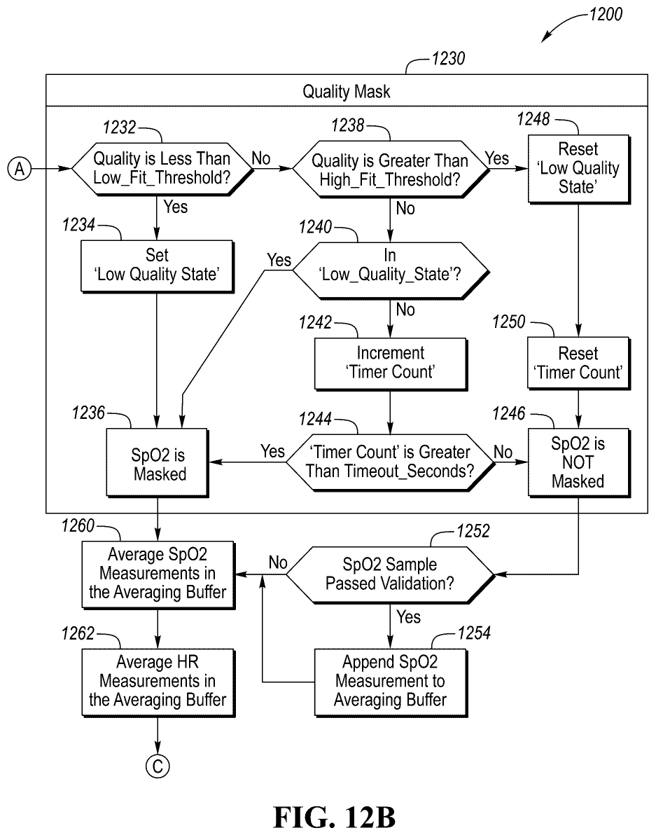

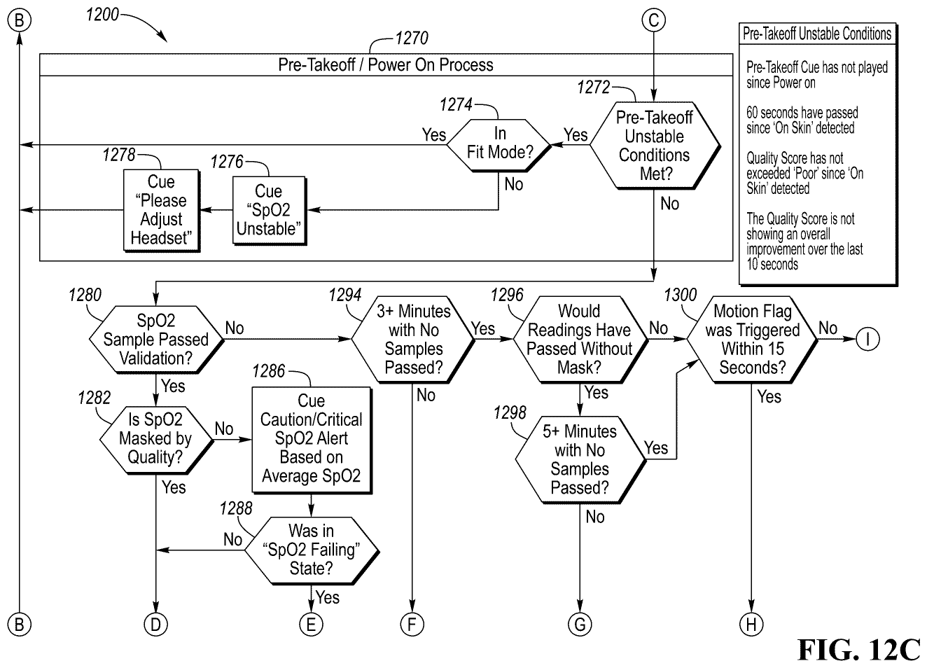

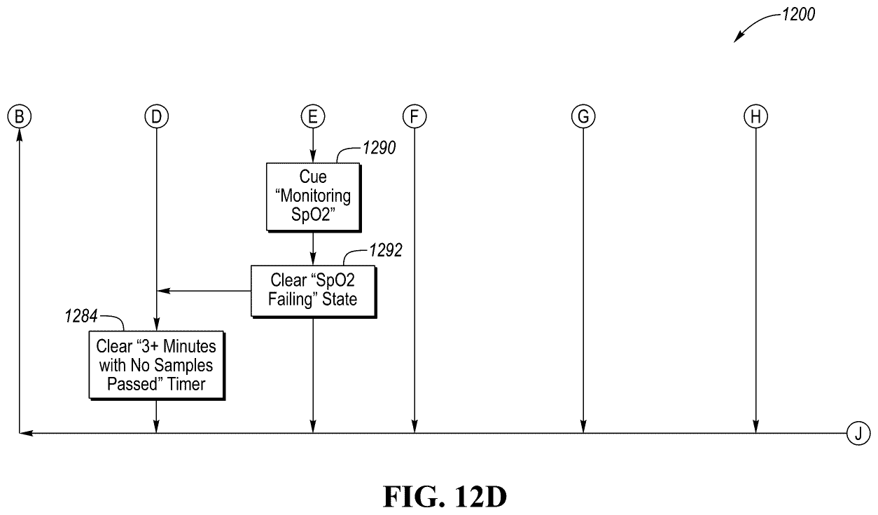

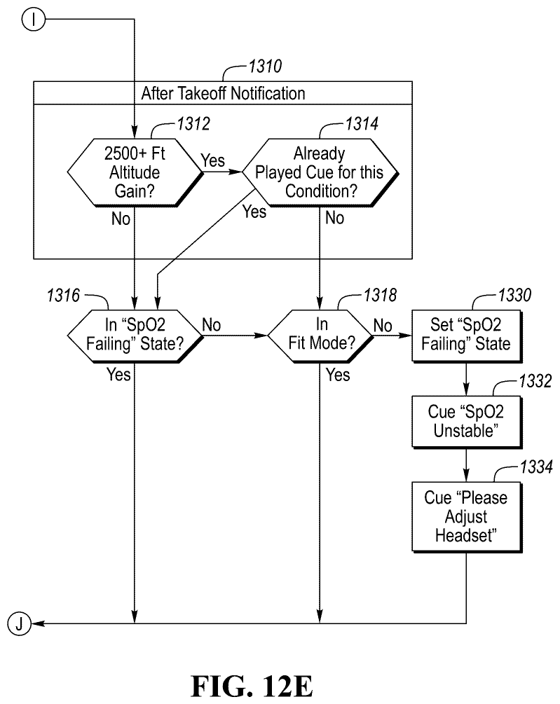

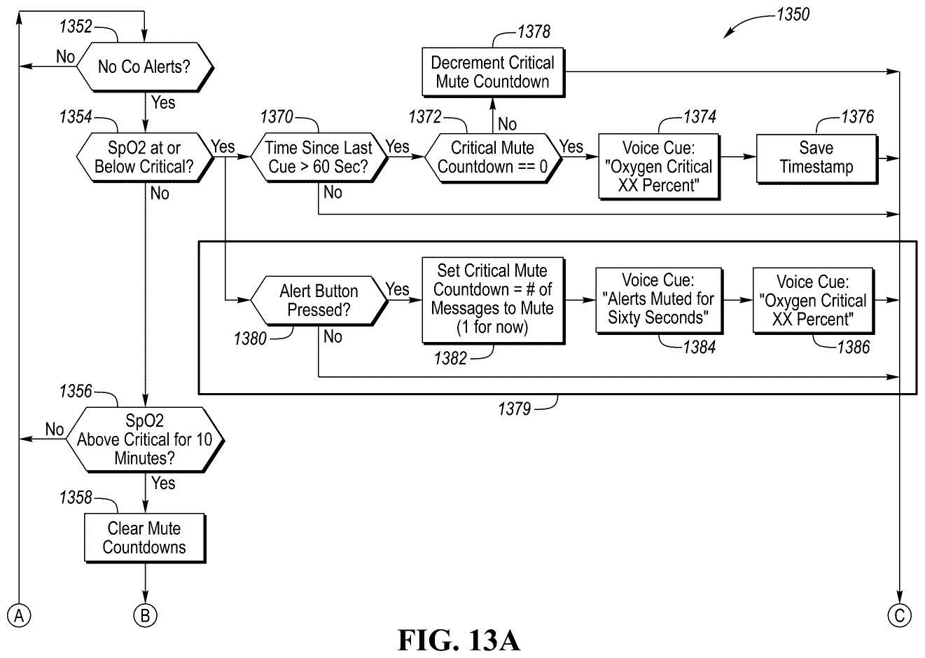

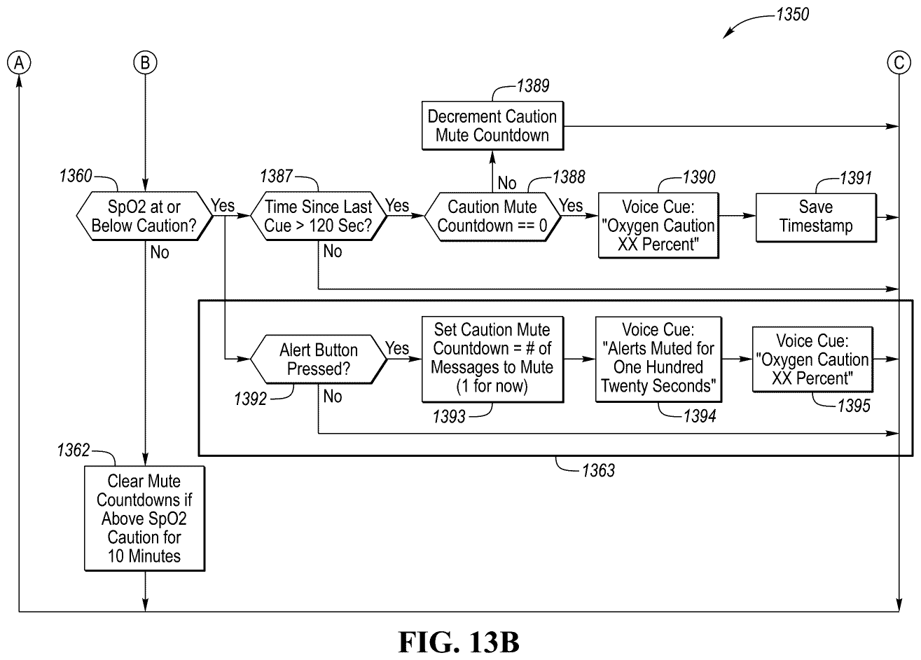

FIGS. 11-13 are block diagrams illustrating operation of a representative control system for a circumaural headset having a formable cuff sensor with an integrated physiologic sensor according to one or more embodiments. System 1100 includes a controller 1110, which may include a processor 1112. As those of ordinary skill in the art will recognize, a controller 1110 may refer to software and/or hardware that cooperate to provide control of the system. Controller 1110 and/or processor 1112 may be implemented by general purpose or special purpose processors, chips, or microcontrollers, that may include one or more programmable circuits, elements, microprocessors, etc., such as digital signal processors (DSPs), FPGAs, and ASICs, for example. Controller 1110 communicates with sensor hub of physiologic sensor 1114, speaker/driver 1116, and microphone 1118 via wired and/or wireless communication. Controller(s) 1110 may be programmed to perform various functions, features, or algorithms as generally described herein and as represented by flow charts or similar diagrams such as shown in FIGS. 12-13. Various steps or functions illustrated may be distributed across two or more controllers in communication with one another.

FIGS. 12A-12E and FIGS. 13A-13B are flowcharts illustrating operation of a system or method for controlling a circumaural headset having an adjustable physiologic sensor according to one or more embodiments in an aviation application. The flowcharts provide representative control strategies and/or logic that may be implemented using one or more processing strategies such as event-driven, interrupt-driven, multi-tasking, multi-threading, and the like. Various control strategies including, but not limited to, open-loop, closed-loop, adaptive, feedback, feedforward, and hybrid strategies may be implemented by control logic, functions, or software executed by controller 1110 or distributed among one or more controllers or processors to provide active noise reduction, processing of sensor signals or associated data provided by a sensor hub to monitor physiological conditions and/or movements of the user, environmental or ambient conditions, and/or processing or analysis of sensor signals or associated data to provide an alert or control signal to a local or remote device, such as a microphone or speaker, in various embodiments. Alternatively, sensor data may be transmitted for storage and/or processing at a remote computer, server, or cloud device, for example.

Various steps or functions illustrated may be performed in the sequence illustrated, in parallel, or in some cases omitted. Although not always explicitly illustrated, one of ordinary skill in the art will recognize that one or more of the illustrated steps or functions may be repeatedly performed depending upon the particular processing strategy being used. Similarly, the order of processing is not necessarily required to achieve the features and advantages described herein, but is provided for ease of illustration and description. The control logic may be implemented primarily in software executed by one or more microprocessor-based controllers having associated memory and circuitry as generally represented by controller(s) 1110 and microprocessor(s) 1112. The control logic, strategy, or algorithm may be implemented in software, hardware, or a combination of software and hardware in one or more controllers depending upon the particular application. When implemented in software, the control logic may be provided in one or more non-transitory computer-readable storage devices or media having stored data representing code or instructions executed by a processor to perform the described function or feature. The computer-readable storage devices or media may include one or more of a number of known physical devices which utilize electric, magnetic, and/or optical storage to keep executable instructions and associated information, operating variables, and the like.

Control strategy 1200 is initialized in an “SpO₂ Failing” state and will exit this state when the Quality Mask 1230 (FIG. 12B) is lifted. The “SpO₂ Failing” state indicates when the quality score is improved to 80 after startup or after entering “Failing” state. The strategy then monitors output from the MAX32664 sensor hub for physiologic sensor data including heart rate (HR) and blood oxygen saturation (SpO₂) as represented at 1210 in addition to various related flags that may provide an indication of the quality of measurements or potential issues/corrections to improve measurement quality.

In addition to physiologic sensor data related to HR and SpO₂ the sensor hub provides sensor-related data or flags as previously described. Sensor-related data or flags may include a motion flag to indicate excessive motion as determined by an accelerometer as accurate SpO₂ measurements can only be achieved if the user is at rest with little or no motion. If the motion flag is set, the sensor algorithm repeats the last calculated SpO₂ value for 15 seconds if the last report SpO₂ value is higher than 94%, with no output after 15 seconds. A Low PI flag indicates that red or infrared (IR) perfusion index (PI) is too low (below 0.05%). If the low PI flag is set, the algorithm repeats the last calculated SpO₂ for 15 seconds only if the last reported value is higher than 94% and after 15 seconds no output is provided because accurate SpO₂ measurements cannot be achieved with a red or IR PI below 0.05%. The PI threshold may be configurable in some applications. An Unreliable R flag indicates unreliable measurements from red and IR signals, which may be due to low signal quality caused by out of range contact force between the sensor and user. If the Unreliable R flag is set, the algorithm repeats the last calculated SpO₂ value only if the last reported value is higher than 94% and after 15 seconds no output is provided. A Low Signal Quality (SNR) flag is a combination of the Low PI flag and Unreliable R flags. If Low SNR flag is set, the algorithm repeats the last calculated SpO₂ value for 15 seconds only if the last reported value is higher than 94% and after 15 seconds no output is provided. An Unreliable Orientation flag indicates whether the measurement position is reliable or not but does not affect the SpO₂ reporting. A Confidence Level provides an indication of the confidence of the measurement and is reported as a value between 0-100%.

Block 1212 determines whether HR data is valid. If yes, the HR measurement is saved to an associated averaging buffer as indicated at 1214. Otherwise, an invalid HR counter is incremented as indicated at 1216. Block 1218 determines whether the SpO₂ data is valid. If no, then an invalid SpO₂ counter is incremented at 1220. Otherwise, the SpO₂ validation state is saved as indicated at 1222. A quality score is generated at block 1224 and processing continues with the Quality Mask 1230 (FIG. 12B) as indicated by connector “A”.

As shown in FIG. 12B, Quality Mask 1230 determines whether quality is less than a configurable low-fit threshold at 1232, with a representative value of 50, for example. If yes, a corresponding flag is set at 1234 and the SpO₂ data is masked at 1236. If no, then block 1238 determines if quality is greater than a configurable high-fit threshold at 1238, with a representative value of 80, for example. If no, then block 1240 determines whether the low quality state flag is set. If yes, then SpO₂ is masked as indicated at 1236. If no, then an associated timer counter is incremented as indicated at 1242. Block 1244 determines whether the timer counter exceeds an associated threshold at block 1244. If yes, the SpO₂ is masked as indicated at 1236. If no, then SpO₂ is not masked as indicated at 1246. Similarly, if block 1238 determines that quality exceeds the high-fit threshold, then the low quality state flag is set at 1248, the timer count is reset at 1250, and SpO₂ is not masked as indicated at 1246 and processing continues at 1252 to determine whether the SpO₂ data passed validation. If yes, the measurement is added to the averaging buffer at 1254 and block 1260 determines an average SpO₂ from samples measurements that passed validation and were recorded in the averaging buffer. Masked values may be stored or recorded in a different buffer for subsequent use as described in greater detail with respect to FIG. 12C. Block 1262 then determines an average of HR measurements in the averaging buffer associated with the validated SpO₂ measurements. Control then continues with the Pre-takeoff/Power On Process 1270 at connector “C” of FIG. 12C.

Block 1272 of FIG. 12C determines whether Pre-Takeoff Unstable conditions are met which include that the Pre-Takeoff Cue has not played since power-on, 60 seconds have passed from an “On Skin” detection corresponding to contact detected between the physiologic sensor and skin of the user, Quality Score has not exceeded “Poor” since “On Skin” detected, and the Quality Score is not improving over the previous 10 seconds. If the conditions are met as indicated at 1272, then block 1274 determines whether Fit Mode is active. If yes, control returns to block 1210 of FIG. 12A as indicated by connector “B”. If Fit Mode is not active, then block 1276 cues an audio alert for “SpO₂ Unstable” and block 1278 cues an audio alert for “Please Adjust Headset.” Audio alerts may be played by the speaker/driver of a designated one or both earcups. If the Pre-Takeoff Unstable conditions are not met as determined at block 1272, then block 1280 determines whether the SpO₂ sample passed validation (using metrics other than the Quality Mask and Motion flags). Representative SpO₂ validation metrics may include examination of various signal quality metrics determined by the MAX32664 sensor hub such as SpO₂ confidence>40%, SpO₂ low signal is False (i.e. pass), Low Perfusion Index (PI) is False (i.e. pass), SpO₂ unreliable R is False (i.e. pass), and Skin Detection shows that contact with skin is valid. The validation flag is set if all the preceding conditions are met and block 1282 determines whether SpO₂ is masked by quality. If yes, then block 1284 (FIG. 12D) clears the timer associated with 3 minutes or more with no samples passed. If no, then block 1286 cues a caution/critical SpO₂ alert based on average SpO₂ and block 1288 determines whether SpO₂ failing state was active. If yes, then block 1290 (FIG. 12D) cues the “Monitoring SpO₂ ” audio alert, block 1292 clears the “SpO₂ failing” state, and block 1284 (FIG. 12D) clears the “3+minutes with no samples passed” timer. Processing then returns to block 1210 (FIG. 12A) as represented by connector “B”.

Block 1294 provides additional validation metrics other than the quality mask and motion detection by determining whether 3 or more minutes have passed with no samples passed. If no, processing returns to block 1210 (FIG. 12A) as represented by connector “B” (FIG. 12D). If yes, block 1296 determines whether data or samples would have passed without the quality mask and may include a counter for the number of masked values. If yes, block 1298 determines whether 5 or more minutes have elapsed with no samples passed. If no, processing returns to block 1210 (FIG. 12A) as represented by connector “B” (FIG. 12D). If yes, block 1300 determines whether the motion flag was triggered within the last 15 seconds. If yes, processing returns to block 1210 (FIG. 12A) as represented by connector “B” (FIG. 12D). If no, processing continues with After Takeoff Notification 1310 (FIG. 12E) where block 1312 determines whether 2500 feet (762 m) or more of altitude gain has occurred, which is a representative altitude gain indicative of lower task load for a pilot after takeoff before generating an audio alert. As also shown in FIG. 12E, if yes, block 1314 determines whether an audio cue has already been played for this condition. If yes at block 1314, or if no at block 1312, then processing continues at block 1316, which determines whether the SpO₂ failing state flag is set. If no at block 1316, or if no at block 1314, then block 1318 determines whether fit mode is active. If yes at block 1316, or if yes at block 1318, processing returns via connector “J” to block 1210 (FIG. 12A) as represented by connector “B” (FIG. 12D).

With continuing reference to FIG. 12E, if block 1318 determines that Fit Mode is not active, then block 1330 sets the “SpO₂ failing” state flag. Block 1332 cues an “SpO₂ Unstable” alert and block 1334 cues a “Please Adjust Headset” alert before processing returns via connector “J” to block 1210 (FIG. 12A) as represented by connector “B” (FIG. 12D).

As illustrated in FIGS. 13A and 13B, control strategy, process, or algorithm 1350 monitors for any carbon monoxide (CO) alerts at 1352 and does not proceed if there is an active CO alert. If there are no CO alerts at 1352, then block 1354 determines whether SpO₂ is at or below a critical threshold. The critical threshold may be user-specified within a predetermined range, which is 80%-90% with a default threshold value of 85% in one embodiment. If no, then block 1356 determines whether SpO₂ has been above the critical threshold for 10 minutes. If no, then processing returns to block 1352. If yes, then block 1358 clears alert mute countdowns. Block 1360 (FIG. 13B) determines if SpO₂ is at or below a caution threshold. The caution threshold may be user-specified within a predetermined range, which is 88%-98% with a default threshold value of 92% in one embodiment. If no, then block 1362 (FIG. 13B) clears the alert mute countdowns if SpO₂ remains above the caution threshold for 10 minutes.

If block 1354 is yes, then block 1370 determines if the elapsed time from the last cue exceeds 60 seconds. If no, the process returns to block 1352 via connector “C” and connector “A” (FIG. 13B). If yes, block 1372 determines whether the critical mute countdown has reached zero. If no, block 1378 decrements the critical mute countdown and the process returns to block 1352 via connector “C and connector “A” (FIG. 13B). If yes, block 1374 generates a voice cue for “Oxygen Critical xx Percent” and block 1376 records an associated timestamp with processing continuing to block 1352 via connector “C” and connector “A” (FIG. 13B).

If block 1354 is yes, then alert mute processing 1379 is executed beginning with determining whether an alert mute button has been pressed at block 1380. If no, processing continues with block 1352. If yes, block 1382 sets the critical mute countdown to an initial configurable value, which may be a value of unity. Block 1384 then cues an “Alerts Muted for Sixty Seconds” message and block 1386 cues “Oxygen Critical xx Percent” alert at 1386 before processing returns to block 1352 via connector “C” and connector “A” (FIG. 13B). As shown in FIG. 13B, a similar mute alert processing block 1363 is executed if block 1360 determines that SpO₂ is at or below the caution threshold with block 1392 determining whether an alert button is pressed. If no, processing continues to block 1352 via connector “C” and connector “A”. If yes at block 1392, block 1393 sets the caution mute countdown to a configurable number of messages to mute with a representative default value of unity. Block 1394 then cues an “Alerts Muted for One-hundred-twenty Seconds” message and block 1395 cues a voice alert for “Oxygen Caution xx Percent.” Processing then continues to block 1352 via connector “C” and connector “A”.

As shown in FIG. 13B, if block 1360 is yes, block 1387 determines whether elapsed time from the last cue exceeds 120 seconds. If no at block 1387, processing returns to block 1352 via connector “C” and connector “A”. If yes at block 1387, block 1388 determines whether the caution mute countdown has reached zero. If no at block 1388, block 1389 decrements the caution mute countdown and processing returns to block 1352 via connector “C” and connector “A”. If yes at block 1388, then block 1390 generates a voice cue for “Oxygen Caution xx Percent” and block 1391 records an associated timestamp before processing returns to block 1352 via connector “C” and connector “A”.

As demonstrated by the representative embodiments illustrated and described in this disclosure, one or more advantages may be provided. For example, adjustable mounting of a physiologic sensor within a circumaural headset may allow the user to adjust the position of the sensor relative to the headset to improve signal to noise ratio (SNR) and resulting accuracy and reliability of the sensor signal. The circumaural headset may provide isolation for the physiologic sensor to reduce the effect of environmental factors, such as ambient noise and light, on the sensor signals. Resilient mounting of a physiologic sensor may improve skin contact with the sensor during physical activity, while also improving comfort. Positioning of a physiologic sensor in contact with the skin in front of the tragus over at least a portion of the TMJ provides a viable location for measurement of various physiologic parameters, such as heartrate, oxygen saturation, blood flow, etc. In addition, positioning of the sensor forward of the tragus using a circumaural headset/headphone provides limited location variability from person to person. An adjustable physiologic sensor mount according to various embodiments facilitates user adjustment and positioning of the sensor in two dimensions for proper placement with a third-dimension adjustment for comfort and proper skin contact. Detection of jaw movement using a physiologic sensor may be used to provide an automatic muting or gating function for a communication microphone associated with the headset, or to provide local or remote alerts based on inferred behavior associated with jaw position or movements.

While representative embodiments are described above, it is not intended that these embodiments describe all possible forms of the claimed subject matter. The words used in the specification are words of description rather than limitation, and it is understood that various changes may be made without departing from the spirit and scope of the disclosure. Additionally, the features of various implementing embodiments may be combined to form further embodiments that may not be illustrated or described in combination. While the best mode has been described in detail, those familiar with the art will recognize various alternative designs and embodiments within the scope of the following claims. While various embodiments may have been described as providing advantages or being preferred over other embodiments with respect to one or more desired characteristics, those of ordinary skill in the art will recognize that one or more characteristics may be compromised to achieve desired system attributes, which depend on the specific application and implementation. These attributes include, but are not limited to: cost, strength, durability, life cycle cost, marketability, appearance, packaging, size, serviceability, weight, manufacturability, ease of assembly, etc. Any embodiments described as less desirable than other embodiments or prior art implementations with respect to one or more characteristics are not outside the scope of the disclosure and may be desirable for particular applications.

1. A headset comprising:
a circumaural earcup assembly having an earcup with a faceplate and an ear seal mounted to the faceplate, the earcup having a socket secured within an interior of the earcup and configured to receive a plug; and
a cuff having a formable wireframe encapsulated in a molded material, the cuff including side portions formable to grip corresponding sides of the ear seal, the side portions connected by a center portion having an opening, the cuff including an integrated physiologic parameter sensor extending at least partially through the opening, the integrated physiologic parameter sensor configured for coupling by a connector to at least one processor programmed to process sensor data, the connector including a cable terminating in a plug configured to engage the socket in the earcup to removably secure the cuff to the earcup.

2. The headset of claim 1 wherein the cuff comprises an outer cuff formed of molded silicone of unitary construction.

3. The headset of claim 2 wherein the cuff further comprises an inner cuff formed of molded silicone encapsulating the formable wireframe, the inner cuff fixedly secured to an underside of the outer cuff.

4. The headset of claim 3 wherein the center portion of the outer cuff tapers from thicker to thinner toward upper and lower edges of the center portion.

5. The headset of claim 4 wherein a center portion of the inner cuff tapers from thicker to thinner toward upper and lower edges of the center portion of the inner cuff.

6. The headset of claim 1 wherein the physiologic parameter sensor comprises a pulse oximetry sensor.

7. The headset of claim 6 wherein the plug of the connector comprises a retainer configured to cooperate with a complementary retainer of the socket in the earcup configured to provide mechanical coupling of the plug and the socket.

8. The headset of claim 7 wherein the retainer of the socket is integrally molded in the faceplate of the earcup.

9. The headset of claim 3 wherein the physiologic sensor comprises a circuit board and wherein the inner cuff comprises a molded inset configured to recess the circuit board such that the circuit board is sandwiched between the outer cuff and the inner cuff.

10. The headset of claim 9 wherein the inner cuff comprises a molded channel configured to recess a portion of the connector and sandwich the recessed portion of the connector between the inner cuff and the outer cuff.

11. The headset of claim 1 further comprising:
a controller; and
a speaker disposed within the circumaural earcup assembly and coupled to the controller; and
wherein the controller is programmed to generate voice alert signals for the speaker in response to signals from the integrated sensor indicating blood oxygen saturation of a user being below an associated threshold level for a programmable amount of time.

12. The headset of claim 11 wherein the controller is programmed to generate an average blood oxygen saturation value using only blood oxygen saturation measurements from the integrated sensor that are above a first fit threshold and below a second fit threshold.

13. The headset of claim 11 wherein the controller is programmed to suppress alert signals during takeoff.

14. The headset of claim 13 wherein the controller is programmed to suppress alert signals in response to detecting an increase in altitude of less than 2500 feet after powering on.

15. The headset of claim 1 wherein the faceplate of the earcup includes a plurality of sliding position reference marks and wherein at least one side portion of the cuff includes a single alignment mark to facilitate positioning of the cuff relative to the faceplate.

16. The headset of claim 15 further comprising at least one controller programmed to provide feedback to a user in response to signal quality from the integrated sensor at each of a plurality of different relative positions between the cuff and the faceplate.

17. A headset comprising:
a circumaural earcup assembly having an earcup with a faceplate and an ear seal mounted to the faceplate, the earcup having a socket secured within an interior of the earcup and configured to receive a plug;
a cuff having an outer portion formed of molded silicone and a separate inner portion formed of molded silicone, the inner portion encapsulating a formable wireframe and secured by an adhesive to an underside of the outer portion, the cuff including side portions formable to grip corresponding sides of the ear seal, the side portions connected by a center portion having an opening, the cuff including an integrated physiologic parameter sensor extending at least partially through the opening, the integrated physiologic parameter sensor configured for coupling by a connector to at least one processor programmed to process sensor data, the connector including a cable terminating in a plug configured to engage the socket in the earcup to removably secure the cuff to the earcup;
a controller; and
a speaker disposed within the circumaural earcup assembly and coupled to the controller;
wherein the controller is programmed to generate voice alert signals for the speaker in response to signals from the integrated sensor indicating blood oxygen saturation of a user being below an associated threshold level for a programmable amount of time.

18. The headset of claim 17 wherein the controller is further programmed to suppress alert signals when the headset is below a predetermined altitude.

19. The headset of claim 17 wherein the controller is programmed to suppress alert signals in response to detecting an increase in altitude of less than 2500 feet relative to the altitude within a predetermined time of powering on the headset.

20. The headset of claim 17 wherein the controller is further programmed to provide feedback to a user in response to signal quality from the integrated sensor at each of a plurality of different relative positions between the cuff and the faceplate.
